(19) United States
(12) Patent Application Publication
(10) Pub. No.: US 2022/0370644 A1
(43) Pub. Date: Nov. 24, 2022

(54) MYELOID CELL-TARGETED NANOPARTICLES AND RELATED COMPOSITIONS AND METHODS

(71) Applicant: The Board of Trustees of the Leland Stanford Junior University, Stanford, CA (US)

(72) Inventors: Alexander Honkala, Berkeley, CA (US); Bryan Smith, East Lansing, MI (US); Katy Margulis, San Francisco, CA (US); Richard Zare, Stanford, CA (US)

(21) Appl. No.: 17/783,939

(22) PCT Filed: Dec. 16, 2020
(86) PCT No.: PCT/US2020/065342

§ 371 Date: Jun. 9, 2022

Related U.S. Application Data
(60) Provisional application No. 62/949,217, filed on Dec. 17, 2019.

(51) Int. Cl. A61K 49/00; A61K 47/69; A61K 47/64; A61K 31/635; A61K 31/519; A61P 35/00

(52) U.S. Cl. CPC A61K 49/0093; A61K 47/6929; A61K 47/643; A61K 49/0034; A61K 31/635; A61K 31/519; A61P 35/00

(57) ABSTRACT

Provided are targeted nanoparticles. In certain embodiments, the targeted nanoparticles comprise a nanoparticle and a myeloid cell (MC) targeting moiety stably associated with the outer surface of the nanoparticle. According to some embodiments, the MC targeting moiety is an immunosuppressive myeloid cell (isMC) targeting moiety. In certain embodiments, the targeted nanoparticles further comprise a detectable label (e.g., an in vivo imaging agent), a drug, or both. Also provided are compositions comprising the targeted nanoparticles of the present disclosure. Methods of using the targeted nanoparticles to image MCs (e.g., isMCs) and/or to modulate and/or disrupt MCs (e.g., isMCs) are also provided.

### CROSS-REFERENCE TO RELATED APPLICATIONS

This application claims the benefit of U.S. Provisional Patent Application No. 62/949,217, filed Dec. 17, 2019, which application is incorporated herein by reference in its entirety.

### INTRODUCTION

The nature of the myeloid cell response to inflammation, in cancer and other diseases, is a significant determinant of disease outcome and patient health. Myeloid cells are a heterogeneous lineage that include macrophages, dendritic cells, and neutrophils, which mediate inflammatory reactions. Among these are also myeloid-derived suppressor cells (MDSCs), a group of immature myeloid cells that strongly suppress the function of CD8+ T-cells and natural killer (NK) T-cells involved in tumor progression and wound healing. During an acute inflammatory response, as in the case of pathogen invasion, immature myeloid cells quickly expand and differentiate to monocytes and activated neutrophils, whose activity are determined by the immunological milieu in which they are activated. However, during persistent inflammation, as seen in chronic conditions such as many cancers, normal hematopoiesis may be disrupted, resulting in both bone-marrow-based and local myelopoiesis where accumulated immature myeloid cells are exposed to varied differentiation signals and may instead convert into MDSCs or other noncanonical myeloid cell sub-types, such as tolerogenic dendritic cells or “M2” macrophages. These noncanonical myeloid cells exert myriad immunosuppressive functions, which may normally help regulate inflammation at a wound site, but, in the context of cancer, are co-opted to retard the ability of canonical dendritic cells, CD8+ T-cells, and NK T-cells to combat tumor growth and contribute to poor prognosis. Thus, detection of myeloid cell presence and phenotype and/or activity at a tumor site is highly important for diagnostic, prognostic, and treatment decision purposes, while inhibition of myeloid-mediated immunosuppressive functions is a therapeutic strategy with the potential to reduce cancer progression and patient mortality.

Studies attempting to detect, characterize, modulate, and quantify myeloid cells have found that the levels and phenotype of myeloid cells in circulation and in the tumor microenvironment at the time of diagnosis are a significant prognostic indicator for disease progression in breast, colorectal, and other cancers. The presence of MDSCs in the tumor microenvironment also reduces treatment efficacy. Improved detection of myeloid cell levels, phenotype, localization, and activity may have significant impact on treatment design and patient stratification, which demands improved means of measuring, localizing, and eventually reprogramming, myeloid cells in patients.

For detection and quantification of myeloid cells, ex vivo analysis of blood by flow cytometry has conventionally been used, such as to surface receptors of myeloid cells such as Ly6C and CD11b in mice. This strategy enables discrimination of certain types of myeloid cells from other, non-immunosuppressive myeloid cell subpopulations, such as mature neutrophils or macrophages. However, such detection is limited to peripheral blood or terminal samples and as such is not capable of dynamically tracking myeloid cell migration to tumor loci nor modulate their function.

### SUMMARY

Provided are targeted nanoparticles. In certain embodiments, the targeted nanoparticles comprise a nanoparticle and a myeloid cell (MC) targeting moiety stably associated with the outer surface of the nanoparticle. According to some embodiments, the MC targeting moiety is an immunosuppressive myeloid cell (isMC) targeting moiety. In certain embodiments, the targeted nanoparticles further comprise a detectable label (e.g., an in vivo imaging agent), a drug, or both. Also provided are compositions comprising the targeted nanoparticles of the present disclosure. Methods of using the targeted nanoparticles to image MCs (e.g., isMCs) and/or to modulate and/or disrupt MCs (e.g., isMCs) are also provided.

### BRIEF DESCRIPTION OF THE FIGURES

FIG. 1 Nanoparticle (NP) size and loading. Panels A and B: SEM images of (panel A) pristine BSA NPs and (panel B) ICG-loaded NPs. The insets show representative size distribution by DLS. Panel C: ICG-loaded NPs after multiple washes with PBS by centrifugal filtration using 100 kDa MWCO centrifugal filters: 1. ICG is completely retained in NPs, which are accumulated in the pellet having an intense green color; 2. Completely clear filtrate.

FIG. 2 Stability of NPs. Panel A: ICG content in NPs in storage at 4° C. in PBS. Panel B: Cumulative release from NPs at 37° C. in 20% v/v FBS in PBS solution. The NPs do not exhibit an initial “burst release” of payload characteristic of many other nanoparticle formulations.

FIG. 3 Ligand conjugation to albumin NPs. Panel A: Schematic of NP formation process employed: 1. Globular protein BSA and water-soluble ICG are dissolved in water; 2. Resultant aqueous solution is mixed with an excess of ethanol (EtOH); 3. NPs are formed by albumin desolvation and preserved by thermal denaturation of albumin chains. ICG is incorporated into the particles; 4. G-CSF is attached to the particle surface via carbodiimide linkage at pH=5.5 with subsequent replacement of medium to PBS. Stable NPs are formed. Panel B: Representative distributions of hydrodynamic diameter of ICG-loaded NPs before (solid line) and after (dotted line) G-CSF linkage.

FIG. 4 G-CSF-targeted and undecorated albumin NP uptake by RAW264.7 cells. Inclusion of the G-CSF targeting moiety greatly increased NP uptake by RAW264.7 multiple myeloma cells, which express the G-CSF-R receptor. Undecorated control NPs showed limited uptake. Dose amounts are NP mass.

FIG. 5 NP selectivity in mixed splenocytes. Primary immune cells were isolated from the spleens of 4T1 breast cancer-bearing immunocompetent Balb/c mice. 4T1 was implanted in the mammary fat pad via subcutaneous injection and allowed to grow for 10 days before primary cell isolation. In each study, splenocytes were plated at 100,000-250,000 cells/well and treated with varying doses of G-CSF-decorated and undecorated albumin NP bearing ICG to track internalization. After 30 minutes of incubation, cells were washed and prepared for flow cytometry, which measured NP internalization via emission >800 nm as well as several cell surface markers indicative of immune cell subpopulations. Panel A: Incubation of 1 μg FITC-labeled NPs showed that G-CSF decorated and undecorated NP controls both accumulate in MDSCs at much higher levels than in CD3+ T-cells, in which limited nonspecific uptake is seen. Inclusion of the G-CSF targeting moiety significantly increased MDSC uptake. MDSCs were defined as CD11b+/Ly6C+ or CD11b+/Ly6G+ and monocytes were defined as CD14+. Panels B and C: G-CSF decoration again increased NP uptake in MDSCs in a significant and dose-dependent manner, also demonstrating some uptake in CD14+ monocytes. NPs used here were labeled with indocyanine green (ICG), which improved uptake detection. Statistical significance by t-test, *P<0.05 **P<0.01, ***P<0.001.

FIG. 6 NP uptake in vivo. 10 μg of G-CSF-decorated and undecorated albumin NPs were each injected into separate groups of mice. One group of mice was tumor-naïve and the other group had been inoculated with 4T1 cells in the mammary glands 10 days before the experiment, during which they grew to ˜500 mm³. All NPs were labeled with indocyanine-green (ICG), which permitted visualization of NP distribution via in vivo fluorescence imaging and measurement of NP uptake by cell type via flow cytometry post-sacrifice. All samples were taken 3 hours after NP injection. Panel A: Characterization of the spleen showed that NPs were taken up by Ly6C+ monocytes, Ly6G+ granulocytes, and dual-positive Ly6C+/Ly6G+ immature MDSCs, wherein G-CSF decoration significantly enhanced NP uptake. These trends were not observed for undecorated control NP in tumor-bearing mice or for either undecorated or G-CSF-decorated NP in tumor-free mice. Panel B: The same trend was seen in the 4T1 tumor microenvironment, where G-CSF decoration significantly enhanced uptake into each myeloid cell subpopulation. Panel C: In the liver, G-CSF decoration did little to increase NP uptake when compared with undecorated controls. Interestingly, more NPs were taken up by cells in the liver in tumor-free mice than in 4T1-tumor-bearing mice, suggesting that either fewer NPs were available for internalization or tumor growth alters the composition or activation states of myeloid cells within the liver in a manner that alters their propensity to internalize NPs, decorated or not. Statistical significance was performed by t-test; *P<0.05, **P<0.01, ***P<0.001.

FIG. 7 Tumor Microenvironment Analysis. To test the effect of G-CSF decoration of NPs on biodistribution and tumor MDSC accumulation, 10 μg each of ICG alone, blank NP, and G-CSF-NPs in 100 μL PBS was injected into either 4T1 breast carcinoma-bearing or tumor-free mice and the shaved mice were imaged using a >800 nm filter using the Lago X fluorescent imaging system at 0 (T0, left), 15 (T15, center), and 180 (T180, right) minutes after administration. Tumor, spleen, and liver NP accumulation by cell type were assayed by flow cytometry (FIG. 6).

FIG. 8 Celecoxib-NPs reduce ROS. Celecoxib-NP-treated LPS-stimulated RAW cells significantly reduced ROS 47% from control (normalized to 0 change), suggesting inhibited immunosuppression in vitro (P<0.01).

FIG. 9 Mice (N=6 tumors per group) were injected intravenously with Celecoxib-NP, and calipers were used to assess tumor size (x-axis is days; y-axis is in % change). Vehicle-treated murine tumors grew >20% within 3 days, while Celecoxib-NP tumors shrank ˜25%, a significant change, with *=P<0.01. Error bars: s.d.

### DETAILED DESCRIPTION

Before the nanoparticles, compositions and methods of the present disclosure are described in greater detail, it is to be understood that the nanoparticles, compositions and methods are not limited to particular embodiments described, as such may, of course, vary. It is also to be understood that the terminology used herein is for the purpose of describing particular embodiments only, and is not intended to be limiting, since the scope of the nanoparticles, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the nanoparticles, compositions and methods. The upper and lower limits of these smaller ranges may independently be included in the smaller ranges and are also encompassed within the nanoparticles, compositions and methods, subject to any specifically excluded limit in the stated range. Where the stated range includes one or both of the limits, ranges excluding either or both of those included limits are also included in the nanoparticles, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the nanoparticles, compositions and methods belong. Although any nanoparticles, compositions and methods similar or equivalent to those described herein can also be used in the practice or testing of the nanoparticles, compositions and methods, representative illustrative nanoparticles, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present nanoparticles, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the nanoparticles, compositions and methods, which are, for clarity, described in the context of separate embodiments, may also be provided in combination in a single embodiment. Conversely, various features of the nanoparticles, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present nanoparticles, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Targeted Nanoparticles

The present disclosure provides targeted nanoparticles. The targeted nanoparticles comprise a nanoparticle and a myeloid cell (MC) targeting moiety stably associated with the outer surface of the nanoparticle. The targeted nanoparticles find use in a variety of applications, including but not limited to diagnostic applications and therapeutic applications. For example, the targeted nanoparticles find use in diagnostic imaging applications for assessing myeloid cell infiltration into the tumor microenvironment as a marker of diagnosis, prognosis, and/or treatment response. Also by way of example, the targeted nanoparticles may comprise drug molecules for altering myeloid cell behavior in therapeutic applications, e.g., to overcome limitations inherent to previous systemic approaches. Details regarding the targeted nanoparticles of the present disclosure will now be described.

By “nanoparticle” is meant a particle having at least one dimension (e.g., a greatest dimension) in the range of from 1 nanometer (nm) to 1000 nm, from 20 nm to 750 nm, from 50 nm to 500 nm, including from 100 nm to 300 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain embodiments, the nanoparticle (e.g., a spherical or spheroid particle) has a greatest dimension of from 10 to 200 nm, e.g., from 30 to 100 nm. According to some embodiments, the greatest dimension of the nanoparticle (e.g., the diameter in the case of a spherical or spheroid nanoparticle) is greater than 10 nm but 500 nm or less, 450 nm or less, 400 nm or less, 350 nm or less, 300 nm or less, 250 nm or less, 200 nm or less, or 100 nm or less. In certain embodiments, the greatest dimension of the nanoparticle (e.g., the diameter in the case of a spherical or spheroid nanoparticle) is less than 500 nm, but 10 nm or greater, 20 nm or greater, 30 nm or greater, 40 nm or greater, 50 nm or greater, 60 nm or greater, 70 nm or greater, 80 nm or greater, 90 nm or greater, 100 nm or greater, 125 nm or greater, 150 nm or greater, 175 nm or greater, 200 nm or greater, 225 nm or greater, 250 nm or greater, 275 nm or greater, 300 nm or greater, 350 nm or greater, or 400 nm or greater.

The nanoparticle may be made of any suitable material or mixtures thereof. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, insoluble protein, metals (e.g., gold, silver, and/or the like), metalloids, magnetic materials, and any combinations thereof. The nanoparticles may be magnetically responsive, e.g., by virtue of comprising one or more paramagnetic and/or superparamagnetic substances, such as for example, magnetite. Such paramagnetic and/or superparamagnetic substances may be embedded within a matrix of the nanoparticle, and/or may be disposed on an external and/or internal surface of the nanoparticle.

In certain embodiments, the nanoparticle is a protein nanoparticle. By “protein nanoparticle” is meant the nanoparticle comprises, consists essentially of, or consists of, proteins. The terms “protein”, “polypeptide”, and “peptide” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

According to some embodiments, the nanoparticle is a serum protein nanoparticle. “serum protein nanoparticle” is meant the nanoparticle comprises, consists essentially of, or consists of, one or more types of serum proteins. Serum proteins of interest include, but are not limited to, albumin proteins. Albumins are a group of simple proteins found in the body fluids and tissues of animals and in some plant seeds. Unlike globulins, albumins have low molecular weights, are soluble in water, are easily crystallized and contain an excess of acidic amino acids. Serum and plasma albumin is carbohydrate-free and comprises 55-62% of the protein present. Due to its high charge to mass ratio albumin binds water, Ca²⁺, Na⁺, K⁺, fatty acids, bilirubin, hormones and drugs. The main biological function of albumin is to regulate the colloidal osmotic pressure of blood. Human and bovine albumins contain 16% nitrogen and are often used as standards in protein calibration studies. In certain embodiments, the nanoparticle is an albumin protein nanoparticle that comprises, consists essentially of, or consists of, one or more types albumin proteins. According to some embodiments, such a nanoparticles comprises, consists essentially of, or consists of, bovine serum albumin (BSA), human serum albumin (HSA), polymerized bovine serum albumin (pBSA), polymerized human serum albumin (pHSA), recombinant albumin, Albumin-DX LR, and any combination thereof.

When the targeted nanoparticle comprises an albumin protein nanoparticle, such a nanoparticle may be synthesized using any suitable approach. For example, an albumin protein nanoparticle may be prepared desolvation of albumin from aqueous solution into an ethanolic phase and subsequent thermal gelation of the nanoparticles followed by the replacement of aqueous medium with a buffered solution, e.g., phosphate buffered saline (PBS) (1×, pH 7.4). A non-limiting example of such an approach to produce an albumin protein nanoparticle is described in the Experimental section below.

A targeted nanoparticle of the present disclosure further comprises a myeloid cell (MC) targeting moiety stably associated with the outer surface of the nanoparticle. By “myeloid cell targeting moiety” or “MC targeting moiety” is meant a moiety that binds to a molecule on the surface of myeloid cells. Cells of the myeloid lineage develop during the process of myelopoiesis and include granulocytes, monocytes, megakaryocytes, and dendritic cells. Molecules on the surface of myeloid cells to which the MC targeting moiety may bind include, but are not limited to, CCR8, CD1a, CD1c, CD11b, CD11c, CD14, CD15, CD16, CD32, CD33, CD34, CD45, CD64, CD68, CD80, CD86, CD163, CD169, CD206, HLA-DR, and combinations thereof. In certain embodiments, the MC targeting moiety binds to a molecule on the surface of a tumor-infiltrating myeloid cell (e.g., an immunosuppressive myeloid cell (isMC)) characterized by one of the following: CD11b^{hi} HLA-DR^{lo} CD206⁺ CCR8⁺; CD11b^{hi} HLA-DR^{hi}; CD11b^{hi} CD15^{hi}; CD45⁺ HLA-DR^{hi} CD11c^{hi} CD16⁺ CD1c⁻; CD45⁺ HLA-DR^{hi} CD11c^{hi} CD16⁻ CD1c⁺; CD14⁺ CD169⁺ CD163⁺; CD45⁺ CD11b⁺ CD11c⁺ CD68⁺ CD32⁺ CD64⁻ HLA-DR⁻ CD80⁻ CD86⁻; CD33⁺ CD11b⁺ HLA-DR⁻; CD45⁺ CD11b⁺ HLA-DR^{hi} CD11c⁺ CD14⁺ CD86⁺; CD11b⁺ CD14⁺ HLA-DR^{lo} CD33⁺ CD34⁺ CD15⁺; CD11b⁺ CD15⁺ CD66b⁺ MPO⁺ Arg⁺ CD62^{lo} CD54⁺ CXCR2^{lo} CCR7⁺ CXCR3⁺ CXCR4⁺; CD11b⁺ CD15⁺; CD11b⁺ CD14⁺ HLA-DR^{hi}; CD11b⁺ CD14⁻ CD15^{int} HLA-DR⁺; CD11b⁺ CD14⁻ CD15^{hi} HLA-DR^{+/lo}; CD14⁺ CD163⁺ CD206⁺ HLA-DR⁺ IL-4α⁺; CD45⁺ HLA-DR^{hi} CD11c^{hi} CD16⁺ CD1c⁻; CD45⁺ HLA-DR^{hi} CD11c^{hi} CD16⁻ CD1c⁺; CD45⁺ CD33⁺ HLA-DR^{int} CD15⁻ CD16⁻; Lin-1⁻ HLA-DR⁻ CD33⁺ CD11b⁺ CD15⁺; Lin-1⁻ HLA-DR⁻ CD14⁺; CD45⁺ CD11b⁺ CD14⁺ HLA-DR^{lo}; or CD45⁺ CD11b⁺ CD15⁺.

A targeted nanoparticle of the present disclosure may include one or more of a variety of suitable types of MC targeting moieties. In some embodiments, the MC targeting moiety is a polypeptide. Non-limiting examples of polypeptide MC targeting moieties include antibodies, ligands, and the like. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to cell surface molecule, including, but not limited to, Fv, single chain Fv (scFv), Fab, F(ab′)₂, Fab′, (scFv′)₂, and diabodies; chimeric antibodies; monoclonal antibodies, human antibodies, humanized antibodies (e.g., humanized whole antibodies, humanized antibody fragments, etc.); and fusion proteins including an antigen-binding portion of an antibody and a non-antibody protein or fragment thereof, e.g., an antibody Fc region or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

In certain embodiments, the MC targeting moiety is a ligand. For example, the MC targeting moiety may be a ligand for a receptor expressed on the surface of myeloid cells or a subset of interest thereof, e.g., immunosuppressive myeloid cells (isMCs), or the like. According to some embodiments, the MC targeting moiety is a natural or non-natural ligand for granulocyte colony stimulating factor receptor (G-CSFR). For example, in certain embodiments, the MC targeting moiety is granulocyte colony stimulating factor (G-CSF). An example of a G-CSF MC targeting moiety is a human G-CSF polypeptide (e.g., UniProtKB—P09919) or a functional variant thereof that binds to G-CSFR (e.g., a G-CSF fragment, a G-CSF polypeptide that includes one or more amino acid substitutions, and/or the like, that retains the ability to bind G-CSFR).

According to some embodiments, the MC targeting moiety of a targeted nanoparticle of the present disclosure is a small molecule that binds to a molecule on the surface of myeloid cells or a subset of interest thereof, e.g., immunosuppressive myeloid cells (isMCs), or the like. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer.

In certain embodiments, the MC targeting moiety of a targeted nanoparticle of the present disclosure is an aptamer that binds to a molecule on the surface of myeloid cells or a subset of interest thereof, e.g., immunosuppressive myeloid cells (isMCs), or the like. By “aptamer” is meant a short (e.g., from 20 to 60 nucleotides), single-stranded DNA or RNA (ssDNA or ssRNA) molecules that can selectively bind to a specific target, including proteins, peptides, carbohydrates, small molecules, toxins, and live cells. Aptamers assume a variety of shapes due to their tendency to form helices and single-stranded loops. Aptamers that may be employed in the targeted nanoparticles of the present disclosure include existing aptamers known to bind a molecule on the surface of myeloid cells or a subset of interest thereof (e.g., isMCs), or an aptamer engineered to bind to such a molecule, e.g., using a known aptamer engineering approach such as SELEX (systematic evolution of ligands by exponential enrichment).

The targeted nanoparticles of the present disclosure comprise the MC targeting moiety stably associated with the outer surface of the nanoparticle. By “stably associated” is meant a physical association between two entities in which the mean half-life of association is one day or more in PBS at 4° C. In certain aspects, the physical association between the two entities has a mean half-life of one day or more, one week or more, one month or more, including six months or more, e.g., 1 year or more, in PBS at 4° C. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like.

In certain embodiments, the MC targeting moiety is stably associated with the outer surface of the nanoparticle via a linker. Non-limiting examples of suitable linkers include flexible polymeric linkers comprising natural or non-natural polymers. Non-limiting examples include peptides, lipid oligomers, liposaccharide oligomers, peptide nucleic acid oligomers, polylactate, polyethylene glycol (PEG), cyclodextrin, polymethacrylate, gelatin, and oligourea. According to some embodiments, the MC targeting moiety is stably associated with the outer surface of the nanoparticle via a flexible peptide linker. Non-limiting examples of flexible peptide linkers include those comprising glycine and serine (glycine-serine linkers), where the flexibility of such linkers may be tuned based on the inverse relationship between linker stiffness and glycine content. According to some embodiments, the MC targeting moiety is stably associated with the outer surface of the nanoparticle via a poly(ethylene glycol) (or “PEG”) linker. Purified PEG is available commercially as mixtures of different oligomer sizes in broadly or narrowly defined molecular weight (MW) ranges. For example, “PEG 600” typically denotes a preparation that includes a mixture of oligomers having an average MW of 600. Likewise, “PEG 10000” denotes a mixture of PEG molecules (n=195 to 265) having an average MW of 10,000 g/mol.

A variety of suitable approaches are available for stably associating the MC targeting moiety to the outer surface of the nanoparticle via a linker. For example, the surface of a nanoparticle may be functionalized (or “activated”/“derivatized”) with a reactive group to which the linker may bind to become bound to the surface of the nanoparticle. The surface of the nanoparticle may be functionalized with any useful/convenient reactive group, including but not limited to thiol groups (—SH), amine groups (—NH2), carboxyl groups (—COO), and/or the like.

The MC targeting moiety may be stably associated with the outer surface of the nanoparticle by reacting a first portion of a linker molecule with a compatible reactive group on the surface of the nanoparticle, and subsequently reacting a second portion of the linker molecule with a compatible reactive group of the MC targeting moiety. Suitable strategies include those described in Chemistry of Bioconjugates: Synthesis, Characterization, and Biomedical Applications (Narain, Ed.) ISBN-10: 9781118359143; Bioconjugate Techniques (Hermanson) ISBN-10: 0123822394; and the surface modification/functionalization literature.

Functional groups that may be used to stably associate the MC targeting moiety with the outer surface of the nanoparticle include, but are not limited to, active esters, isocyanates, imidoesters, hydrazides, amino groups, aldehydes, ketones, photoreactive groups, maleimide groups, alpha-halo-acetyl groups, epoxides, azirdines, and the like. Reagents such as iodoacetamides, maleimides, benzylic halides and bromomethylketones react by S-alkylation of thiols to generate stable thioether products. For example, at pH 6.5-7.5, maleimide groups react with sulfhydryl groups to form stable thioether bonds. Arylating reagents such as NBD halides react with thiols or amines by a similar substitution of the aromatic halide by the nucleophile. Because the thiolate anion is a better nucleophile than the neutral thiol, cysteine is more reactive above its pKₐ (˜8.3, depending on protein structural context). Thiols also react with certain amine-reactive reagents, including isothiocyanates and succinimidyl esters. The TS-Link series of reagents are available for reversible thiol modification.

With respect to amine reactive groups, primary amines exist at the N-terminus of polypeptide chains and in the side-chain of lysine (Lys, K) amino acid residues. Among the available functional groups in proteins (e.g., peptide linkers, etc.), primary amines are especially nucleophilic, making them ready targets for conjugation with several reactive groups. For example, NHS esters are reactive groups formed by carbodiimide-activation of carboxylate molecules. NHS ester-activated crosslinkers and labeling compounds react with primary amines in physiologic to slightly alkaline conditions (pH 7.2 to 9) to yield stable amide bonds. The reaction releases N-hydroxysuccinimide (NHS). Also by way of example, imidoester crosslinkers react with primary amines to form amidine bonds. Imidoester crosslinkers react rapidly with amines at alkaline pH but have short half-lives. As the pH becomes more alkaline, the half-life and reactivity with amines increases. As such, crosslinking is more efficient when performed at pH 10 than at pH 8. Reaction conditions below pH 10 may result in side reactions, although amidine formation is favored between pH 8-10.

Numerous other synthetic chemical groups will form chemical bonds with primary amines, including but not limited to, isothiocyanates, isocyanates, acyl azides, sulfonyl chlorides, aldehydes, glyoxals, epoxides, oxiranes, carbonates, aryl halides, carbodiimides, anhydrides, and fluorophenyl esters. Such groups conjugate to amines by either acylation or alkylation.

In certain embodiments, the nanoparticle is a protein nanoparticle (e.g., a serum protein nanoparticle, such as an albumin protein nanoparticle), the MC targeting moiety is or comprises a polypeptide (e.g., a polypeptide ligand (e.g., G-CSF, or the like), an antibody, or the like), and the targeted nanoparticle is produced by covalently linking the nanoparticle to the MC targeting moiety using carbodiimide chemistry based on amide bond formation between the polypeptide chains of the nanoparticle and the MC targeting moiety under mild aqueous conditions. According to some embodiments, such a synthesis is performed with a zero-length crosslinker that leaves no residues in the resultant protein. A non-limiting example of such an approach for making the targeted nanoparticle is described in the Experimental section below.

In certain embodiments, a targeted nanoparticle of the present disclosure further comprises a detectable label. Detectable labels that may be employed include, but are not limited to, fluorescent labels, colorimetric labels, chemiluminescent labels, enzyme-linked reagents, multicolor reagents, avidin-streptavidin associated detection reagents, and the like.

According to some embodiments, the detectable label is a fluorescent label. Fluorescent labels are labeling moieties that are detectable by a fluorescence detector. For example, binding of a fluorescent label to an analyte of interest (e.g., myeloid cells, such as immunosuppressive myeloid cells) allow the analyte of interest to be detected by a fluorescence detector. Examples of fluorescent labels include, but are not limited to, fluorescent molecules that fluoresce upon contact with a reagent, fluorescent molecules that fluoresce when irradiated with electromagnetic radiation (e.g., UV, visible light, x-rays, etc.), fluorescent labels that are detectable by photoacoustic imaging, and the like.

In certain embodiments, suitable fluorescent molecules (fluorophores) for labeling include, but are not limited to, indocyanine green (ICG), IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

According to some embodiments, the detectable label is an in vivo imaging agent. The phrase “in vivo imaging” as used herein refers to methods of detecting the targeted nanoparticles (and in turn, myeloid cells (e.g., isMCs, including tumor-infiltrating myeloid cells) to which the targeted nanoparticles are bound) in a whole, live mammal. Optically detectable agents, such as fluorescent agents (e.g., indocyanine green (ICG)), bioluminescent agents (e.g., luciferases, such as nanoluciferases), and radioactively labeled agents may be detected by in vivo imaging. In vivo imaging may be used provide 2-D as well as 3-D images of a mammal or tissues or cells therein. Charge-coupled device cameras, photodiodes, avalanche photodiodes, photomultiplier tubes, CMOS, or 3D tomographers may be used to carry out in vivo imaging. For example, Burdette J E (2008) Journal of Mol. Endocrin. 40: 253-261 reviews the uses of computed tomography, magnetic resonance imaging, ultrasonography, positron emission tomography, single-photon emission computed tomography, etc., for in vivo imaging. Methods for using a detectable label for real-time imaging of luciferase expression in live animals can be readily adapted for use in the subject methods disclosed herein (e.g., Greer L F et al. (2002) Luminescence 17: 43-74). In vivo imaging of fluorescent proteins in live animals is described in, e.g., Hoffman (2002) Cell Death and Differentiation 9:786-789. In some embodiments, in vivo imaging may be performed by detecting a label that emits light at a wavelength designed to penetrate living tissue. Such labels include long wavelength emitting fluorescent dyes or proteins such as infrared and near infrared dyes or proteins including but not limited to dyes or proteins that emit in the range of about 600 nm to about 800 nm, about 650 nm to about 800 nm, or about 700 nm to about 800 nm. Alternatively, labels designed to emit light that penetrates living tissue may include non-fluorescent reagents including but not limited to red-shifted luciferases.

In vivo imaging can also involve computed tomography, magnetic resonance imaging, ultrasonography, positron emission tomography, single-photon emission computed tomography (SPECT) (See Burdette J E (2008) Journal of Mol. Endocrin., 40:253-261 for details). SPECT can also be used with an integrated x-ray CAT (CT) scanner (SPECT/CT) in the subject methods. The information from many in vivo imaging methods as those described above can provide 3D distribution of the nanoparticles (and in turn, myeloid cells) in the subject.

According to some embodiments, the targeted nanoparticle further comprises an in vivo imaging agent, where the in vivo imaging agent is a photoacoustic imaging agent. Photoacoustic imaging (PAI) bridges the traditional depth limits of ballistic optical imaging and the resolution limits of diffuse optical imaging. Using the acoustic waves generated in response to the absorption of pulsed laser light, it provides noninvasive images of absorbed optical energy density at depths of several centimeters with a resolution of ˜100 μm. This versatile and scalable imaging modality has proven useful for molecular imaging, which enables visualization of biological processes with systemically introduced contrast agents. Agents that find use in photoacoustic imaging include those described in Weber et al. (2016) Nature Methods 13:639-650. In certain embodiments, the targeted nanoparticle comprises a photoacoustic imaging agent, and the photoacoustic imaging agent is indocyanine green (ICG), a tricarbocyanine dye that is safe for intravenous administration.

When the targeted nanoparticle further comprises a detectable label, the detectable label may be incorporated into (e.g., embedded in) the nanoparticle, the detectable label may be stably associated with the outer surface of the nanoparticle, or both.

In certain embodiments, a targeted nanoparticle of the present disclosure further comprises a drug. By “drug” is meant a substance (e.g., small molecule, biologic, or the like) that has a therapeutic effect when administered to an individual in need thereof in an effective amount. Drugs that may be employed include, but are not limited to, isMC-modulating drugs. As used herein, an “isMC-modulating drug” is a drug that modulates the function or viability of isMCs in a therapeutically beneficial manner. A non-limiting example of an isMC-modulating drug is an isMC-disrupting drug (that is, a drug that disrupts one or more normal processes, and/or the viability of, isMCs), e.g., an isMC metabolism-disrupting drug. According to some embodiments, the targeted nanoparticle comprises an isMC metabolism-disrupting drug, and the drug is a nonsteroidal anti-inflammatory drug (NSAID), a non-limiting example of which is a cyclooxygenase-2 (COX-2) inhibitor, e.g., celecoxib. In certain embodiments, the targeted nanoparticle comprises an isMC-modulating drug, where the drug is sildenafil.

When a targeted nanoparticle of the present disclosure further comprises a drug, the drug may be releasably incorporated into the nanoparticle, releasably associated with the outer surface of the nanoparticle, stably associated with the outer surface of the nanoparticle, or any combination thereof.

### Compositions

As summarized above, the present disclosure also provides compositions. The compositions comprise targeted nanoparticles of the present disclosure, e.g., any of the targeted nanoparticles having any of the features described in the Targeted Nanoparticles section hereinabove or the Experimental section below, which are incorporated but not reiterated herein for purposes of brevity.

In certain aspects, the compositions include the targeted nanoparticles present in a liquid medium. The liquid medium may be an aqueous liquid medium, such as water, a buffered solution, and the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Pharmaceutical compositions are also provided. The pharmaceutical compositions comprise targeted nanoparticles of the present disclosure, and a pharmaceutically acceptable carrier. The pharmaceutical compositions generally include an effective amount of the targeted nanoparticles. In certain embodiments, the targeted nanoparticles comprise an in vivo imaging agent, and the effective amount is an amount effective for in vivo imaging of the targeted nanoparticles (and in turn, the MCs (e.g., isMCs) to which the nanoparticles are bound) in an individual in need thereof. According to some embodiments, the targeted nanoparticles comprise a drug (e.g., any of the isMC-modulating drugs described above (e.g., isMC-disrupting drugs, such as isMC metabolism-disrupting drugs)), and the effective amount is a therapeutically effective amount of the targeted nanoparticles. By “therapeutically effective amount” is meant a dosage sufficient to produce a desired result, e.g., an amount sufficient to effect beneficial or desired therapeutic (including preventative) results, such as a reduction in cellular proliferation (e.g., via modulation (e.g., disruption) of MCs (e.g., tumor infiltrating isMCs)) in an individual having a cell proliferative disorder, e.g., cancer. An effective amount may be administered in one or more administrations.

The targeted nanoparticles of the present disclosure can be incorporated into a variety of formulations for diagnostic and/or therapeutic administration. More particularly, the targeted nanoparticles can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, emulsions, injections, inhalants and aerosols.

Formulations of the targeted nanoparticles of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the targeted nanoparticles can be administered alone or in appropriate association, as well as in combination, with other diagnostic and/or pharmaceutically-active compounds. The following methods and excipients are merely examples and are in no way limiting.

For oral preparations, the targeted nanoparticles can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The targeted nanoparticles can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the targeted nanoparticles may be prepared in a pH-buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Methods

Also provided by the present disclosure are methods. The methods comprise administering to a subject in need thereof an effective amount of a pharmaceutical composition of the present disclosure. The methods find use in various applications including diagnostic, prognostic, and/or therapeutic applications.

According to some embodiments, provided are methods of imaging myeloid cells (MCs) in a subject. Such methods comprise administering to the subject a pharmaceutical composition comprising the targeted nanoparticles of the present disclosure that comprise an in vivo imaging agent; and imaging MCs (e.g., isMCs) in the subject by in vivo imaging. In certain embodiments, the targeted nanoparticles comprise an isMC targeting moiety. Any of the isMC targeting moieties described elsewhere herein may be employed. In one non-limiting example, the isMC targeting moiety is a ligand for a receptor on the surface of isMCs. For example, the isMC targeting moiety may be G-CSF for binding to G-CSFR on the surface of isMCs to target the nanoparticles to the isMCs. According to some embodiments, the subject comprises a tumor, and the methods comprise assessing infiltration of the MCs (e.g., isMCs) in the microenvironment of the tumor based on the imaging of the isMCs. Such methods may further comprise providing a diagnosis, prognosis, or both, of the subject based on the imaging of the isMCs. In certain embodiments, the targeted nanoparticles further comprise a drug, e.g., any of the isMC-modulating drugs described above (e.g., isMC-disrupting drugs, such as isMC metabolism-disrupting drugs).

Approaches for in vivo imaging (including photoacoustic imaging) which may be applied to the imaging methods of the present disclosure are known and include those described in, e.g., Smith & Gambhir (2017) Chem. Rev. 117(3):901-986; Diao et al. (2015) Angewandte Chemie International Edition 54:49; Sun et al. (2016) Chem. Sci. 7:6203-6207; and elsewhere.

Also provided are methods of modulating MCs in a subject. Such methods comprise administering to the subject a pharmaceutical composition comprising the targeted nanoparticles of the present disclosure that comprise an isMC-modulating drug, e.g., any of the isMC-modulating drugs described above (e.g., isMC-disrupting drugs, such as isMC metabolism-disrupting drugs), and a pharmaceutically acceptable carrier, in an amount effective to modulate MCs in the subject.

Also provided are methods of enhancing an anti-tumor immune response in a subject having a tumor, comprising administering to the subject a pharmaceutical composition comprising the targeted nanoparticles of the present disclosure that comprise an isMC-disrupting drug, e.g., any of the isMC-disrupting drugs described above (e.g., isMC metabolism-disrupting drugs, such as celecoxib, for example), and a pharmaceutically acceptable carrier, in an amount effective to disrupt MCs in the subject.

According to the methods of modulating MCs and/or enhancing an anti-tumor immune response in a subject having a tumor, the targeted nanoparticles may comprise any of the MC- (e.g., isMC-) targeting moieties described elsewhere herein. In one non-limiting example, the targeting moiety is an isMC targeting moiety, a non-limiting example of which is a ligand for a receptor on the surface of isMCs. For example, the targeting moiety may be G-CSF for binding to G-CSFR on the surface of isMCs to target the nanoparticles to the isMCs. In addition, according to the methods of modulating MCs and/or enhancing an anti-tumor immune response in a subject having a tumor, the targeted nanoparticles may further comprise an in vivo imaging agent to enable in vivo imaging of the MCs (e.g., isMCs) for diagnostic and/or prognostic purposes. As such, the present methods of modulating MCs and/or enhancing an anti-tumor immune response in a subject having a tumor may further comprise imaging the MCs (e.g., isMCs) in the subject.

A variety of subjects are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the subject is a human.

In certain embodiments, the subject has a cancer characterized by the presence of a solid tumor, a semi-solid tumor, a primary tumor, a metastatic tumor, or the like. In some embodiments, the subject has a cancer selected from breast cancer, melanoma, lung cancer, colorectal cancer, prostate cancer, glioma, glioblastoma, bladder cancer, endometrial cancer, kidney cancer, leukemia (e.g., acute myeloid leukemia (AML)), liver cancer (e.g., hepatocellular carcinoma (HCC), such as primary or recurrent HCC), non-Hodgkin lymphoma, pancreatic cancer, thyroid cancer, any combinations thereof, and any sub-types thereof.

According to some embodiments, the methods of modulating MCs and/or enhancing an anti-tumor immune response in a subject having a tumor are effective in treating the tumor of the subject. By “treat”, “treating” or “treatment” is meant at least an amelioration of the symptoms associated with a medical condition of the subject (e.g., cell proliferative disorder, e.g., cancer) of the individual, where amelioration is used in a broad sense to refer to at least a reduction in the magnitude of a parameter, e.g. symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

The pharmaceutical composition is administered to the subject in an effective amount. By “effective amount” is meant a dosage sufficient to produce a desired result, e.g., an amount sufficient to effect beneficial or desired diagnostic and/or therapeutic (including preventative) results, such as a reduction in a symptom of cancer, as compared to a control. In some embodiments, an effective amount is sufficient to slow the growth of a tumor, reduce the size of a tumor, and/or the like. An effective amount may be administered in one or more administrations.

The pharmaceutical composition may be administered to the subject using any available method and route suitable for nanoparticle delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the targeted nanoparticles and/or the desired effect. The pharmaceutical compositions may be administered in a single dose or in multiple doses. In some embodiments, the pharmaceutical composition is administered intravenously. In some embodiments, the pharmaceutical composition is administered by injection, e.g., for systemic delivery (e.g., intravenous infusion) or to a local site.

### Kits

As summarized above, the present disclosure also provides kits. In certain embodiments, a subject kit includes any of the targeted nanoparticles or compositions (e.g., pharmaceutical compositions) of the present disclosure, and instructions for targeting the targeted nanoparticles to myeloid cells (MCs, e.g., isMCs). The instructions may be for targeting the targeted nanoparticles to MCs in vitro (e.g., in the case of MCs in culture) or MCs in vivo.

According to some embodiments, provided are kits that comprise a pharmaceutical composition that finds use in practicing any of the methods of imaging MCs in a subject, modulating MCs in a subject, and/or enhancing an anti-tumor immune response in a subject having a tumor. In certain embodiments, a kit of the present disclosure comprises instructions for administering the pharmaceutical composition to a subject having a tumor to assess infiltration of the MCs (e.g., isMCs) in the microenvironment of the tumor by imaging of the nanoparticles (and in turn, the MCs to which the nanoparticles are bound). According to some embodiments, a kit of the present disclosure comprises targeted nanoparticles that comprise an MC-modulating drug, where the instructions of the kit comprise instructions for modulating the MCs (e.g., isMCs) in the subject. In certain embodiments, the targeted nanoparticles comprise an MC-disrupting drug, and the instructions of the kit comprise instructions for disrupting the MCs (e.g., isMCs) in the subject. According to some embodiments, the targeted nanoparticles comprise an MC-disrupting drug, and the instructions of the kit comprise instructions for administering the pharmaceutical composition to a subject having a tumor to enhance an anti-tumor immune response in the subject.

Such kits may include a quantity of the pharmaceutical composition, present in unit dosages, e.g., ampoules, or a multi-dosage format. As such, in certain embodiments, the kits may include one or more (e.g., two or more) unit dosages (e.g., ampoules) of the pharmaceutical composition. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular targeted nanoparticles employed, the effect to be achieved, and the pharmacodynamics associated with the targeted nanoparticles, in the individual. In yet other embodiments, the kits may include a single multi dosage amount of the pharmaceutical composition.

Components of the kits may be present in separate containers, or multiple components may be present in a single container.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:

1. A targeted nanoparticle comprising:
a nanoparticle; and
a myeloid cell (MC) targeting moiety stably associated with the outer surface of the nanoparticle.
   2. The targeted nanoparticle of embodiment 1, wherein the greatest dimension of the nanoparticle is from 10 to 200 nm.
   3. The targeted nanoparticle of embodiment 1, wherein the greatest dimension of the nanoparticle is from 30 to 100 nm.
   4. The targeted nanoparticle of any one of embodiments 1 to 3, wherein the nanoparticle is spherical or spheroid.
   5. The targeted nanoparticle of any one of embodiments 1 to 4, wherein the nanoparticle is a protein nanoparticle.
   6. The targeted nanoparticle of embodiment 5, wherein the nanoparticle is a serum protein nanoparticle.
   7. The targeted nanoparticle of embodiment 6, wherein the nanoparticle is an albumin protein nanoparticle.
   8. The targeted nanoparticle of embodiment 7, wherein the albumin protein nanoparticle comprises an albumin protein selected from the group consisting of: bovine serum albumin (BSA), human serum albumin (HSA), polymerized bovine serum albumin (pBSA), polymerized human serum albumin (pHSA), recombinant albumin, Albumin-DX LR, and any combination thereof.
   9. The targeted nanoparticle of any one of embodiments 1 to 8, wherein the MC targeting moiety is selected from the group consisting of: a polypeptide, an antibody, a ligand, an aptamer, a nanoparticle, and a small molecule.
   10. The targeted nanoparticle of any one of embodiments 1 to 9, wherein the MC targeting moiety binds to a molecule on the surface of MCs.
   11. The targeted nanoparticle of embodiment 10, wherein the MC targeting moiety binds to a receptor on the surface of MCs.
   12. The targeted nanoparticle of any one of embodiments 1 to 11, wherein the MC targeting moiety is an immunosuppressive myeloid cell (isMC) targeting moiety.
   13. The targeted nanoparticle of embodiment 12, wherein the isMC targeting moiety is a ligand.
   14. The targeted nanoparticle of embodiment 13, wherein the isMC targeting moiety is granulocyte-colony stimulating factor (G-CSF).
   15. The targeted nanoparticle of any one of embodiments 1 to 14, wherein the MC targeting moiety is stably associated with the outer surface of the nanoparticle via an amide bond.
   16. The targeted nanoparticle of any one of embodiments 1 to 15, further comprising a detectable label.
   17. The targeted nanoparticle of embodiment 16, wherein the detectable label is an in vivo imaging agent.
   18. The targeted nanoparticle of embodiment 17, wherein the in vivo imaging agent is a near-infrared (NIR) imaging agent.
   19. The targeted nanoparticle of embodiment 17, wherein the in vivo imaging agent is a photoacoustic imaging agent.
   20. The targeted nanoparticle of any one of embodiments 17 to 19, wherein the in vivo imaging agent is indocyanine green (ICG).
   21. The targeted nanoparticle of any one of embodiments 16 to 20, wherein the detectable label is incorporated into the nanoparticle.
   22. The targeted nanoparticle of any one of embodiments 16 to 21, wherein the detectable label is stably associated with the outer surface of the nanoparticle.
   23. The targeted nanoparticle of any one of embodiments 1 to 22, further comprising a drug.
   24. The targeted nanoparticle of embodiment 23, wherein the drug is a small molecule drug.
   25. The targeted nanoparticle of embodiment 23 or embodiment 24, wherein the drug is an isMC-modulating drug.
   26. The targeted nanoparticle of embodiment 25, wherein the drug is an isMC-disrupting drug.
   27. The targeted nanoparticle of embodiment 26, wherein the drug is an isMC metabolism-disrupting drug.
   28. The targeted nanoparticle of embodiment 27, wherein the drug is a nonsteroidal anti-inflammatory drug (NSAID).
   29. The targeted nanoparticle of embodiment 28, wherein the NSAID is a cyclooxygenase-2 (COX-2) inhibitor.
   30. The targeted nanoparticle of embodiment 29, wherein the COX-2 inhibitor is celecoxib.
   31. The targeted nanoparticle of embodiment 25, wherein the drug is sildenafil.
   32. The targeted nanoparticle of any one of embodiments 23 to 31, wherein the drug is releasably incorporated into the nanoparticle.
   33. The targeted nanoparticle of any one of embodiments 23 to 32, wherein the drug is releasably associated with the outer surface of the nanoparticle.
   34. A composition comprising targeted nanoparticles of any one of embodiments 1 to 33.
   35. The composition of embodiment 34, wherein the composition is a pharmaceutical composition comprising the targeted nanoparticles and a pharmaceutically acceptable carrier.
   36. The composition of embodiment 35, wherein the pharmaceutical composition comprises targeted nanoparticles of any one of embodiments 17 to 22.
   37. A method of imaging myeloid cells (MCs) in a subject, comprising:
administering to the subject the pharmaceutical composition of embodiment 36 in an amount effective to image MCs in the subject; and
imaging MCs in the subject by in vivo imaging.
   38. The method according to embodiment 37, wherein the targeted nanoparticles comprise an isMC targeting moiety.
   39. The method according to embodiment 38, wherein the isMC targeting moiety is a ligand.
   40. The method according to embodiment 39, wherein the isMC targeting moiety is G-CSF.
   41. The method according to any one of embodiments 38 to 40, wherein the subject comprises a tumor, and wherein the method comprises assessing infiltration of the isMCs in the microenvironment of the tumor based on the imaging of the isMCs.
   42. The method according to embodiment 41, further comprising providing a diagnosis, prognosis, or both, of the subject based on the imaging of the isMCs.
   43. The method according to any one of embodiments 37 to 42, wherein the pharmaceutical composition comprises the targeted nanoparticles of any one of embodiments 19 to 22, and wherein the in vivo imaging comprises photoacoustic imaging.
   44. The method according to any one of embodiments 37 to 43, wherein the targeted nanoparticles further comprise a drug as defined in any one of embodiments 23 to 33.
   45. A method of modulating MCs in a subject, comprising administering to the subject a pharmaceutical composition comprising:
targeted nanoparticles of any one of embodiments 25 to 33; and
a pharmaceutically acceptable carrier,
in an amount effective to modulate MCs in the subject.
   46. A method of enhancing an anti-tumor immune response in a subject having a tumor, comprising administering to the subject a pharmaceutical composition comprising:
targeted nanoparticles of any one of embodiments 26 to 33; and
a pharmaceutically acceptable carrier,
in an amount effective to disrupt MCs in the subject.
   47. The method according to embodiment 45 or embodiment 46, wherein the MCs are isMCs and the targeted nanoparticles comprise an isMC targeting moiety.
   48. The method according to embodiment 47, wherein the isMC targeting moiety is a ligand.
   49. The method according to embodiment 48, wherein the isMC targeting moiety is G-CSF.
   50. The method according to any one of embodiments 45 to 49, wherein the targeted nanoparticles comprise a detectable label as defined in any one of embodiments 17 to 22.
   51. The method according to embodiment 50, further comprising imaging the MCs in the subject.
   52. A kit, comprising:
the composition of embodiment 34; and
instructions for targeting the targeted nanoparticles to MCs.
   53. A kit, comprising:
the pharmaceutical composition of embodiment 35; and
instructions for administering the pharmaceutical composition to a subject to target the targeted nanoparticles to MCs in the subject.
   54. The kit of embodiment 53, comprising targeted nanoparticles of any one of embodiments 17 to 22, wherein the instructions comprise instructions for imaging the MCs in the subject.
   55. The kit of embodiment 53 or embodiment 54, wherein the MCs are isMCs and the targeted nanoparticles comprise an isMC targeting moiety.
   56. The kit of embodiment 55, wherein the isMC targeting moiety is a ligand.
   57. The kit of embodiment 56, wherein the isMC targeting moiety is G-CSF.
   58. The kit of any one of embodiments 55 to 57, wherein the instructions comprise instructions for administering the pharmaceutical composition to a subject having a tumor to assess infiltration of the isMCs in the microenvironment of the tumor based on the imaging of the isMCs.
   59. The kit of any one of embodiments 55 to 58, comprising targeted nanoparticles of any one of embodiments 25 to 33, and wherein the instructions comprise instructions for modulating the isMCs in the subject.
   60. The kit of embodiment 59, comprising targeted nanoparticles of any one of embodiments 26 to 33, and wherein the instructions comprise instructions for disrupting the isMCs in the subject.
   61. The kit of embodiment 60, wherein the instructions comprise instructions for administering the pharmaceutical composition to a subject having a tumor to enhance an anti-tumor immune response in the subject.

The following examples are offered by way of illustration and not by way of limitation.

### EXPERIMENTAL

The present examples demonstrate the development of targeted nanoparticles that specifically label myeloid cells throughout the body to monitor their recruitment into the tumor microenvironment. Recombinant granulocyte colony-stimulating factor (G-CSF), a glycoprotein that regulates the migration, proliferation, and functional maintenance of all myeloid cells, was employed as a surface ligand to target the nanoparticles to myeloid cells. This approach enables a preferential accumulation of the nanoparticles in myeloid lineage cells and not in other types of cells within the complex tumor microenvironment. Measuring the localization of the myeloid cells in the tumor microenvironment via this approach will provide insight into the prognostic outlook of cancer patients while providing a platform to deliver pharmacological agents to these cells and modify their behavior to therapeutically alter disease progression.

### Example 1—Nanoparticle Formation, Loading and Characterization

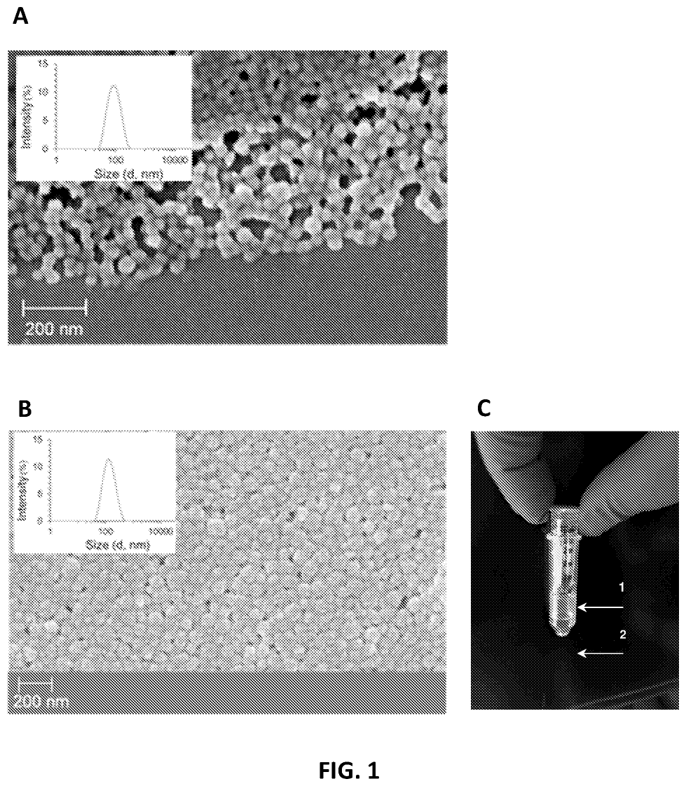

Nanoparticles (NPs) comprising bovine serum albumin were prepared to deliver fluorophores to myeloid cells. The most abundant protein in mammalian blood, albumin, can be used to form NPs with an excellent in vivo safety profile. In this study, NPs were prepared by desolvation of albumin from aqueous solution into an ethanolic phase and subsequent thermal gelation of the NPs followed by the replacement of aqueous medium with phosphate buffered saline (PBS) (1×, pH 7.4). The unloaded NPs had a hydrodynamic diameter of 80±3 nm, determined from the intensity distribution by dynamic light scattering (DLS), and a negative ζ-potential of −15.1±0.6 kV, measured in 0.9% NaCl water solution. Measuring particle size by Scanning Electron Microscopy (SEM) showed that individual dried NP sizes are smaller than those measured by DLS in solution, roughly 30-50 nm in diameter (FIG. 1, panel A). Albumin NPs were first formed by utilizing albumin-fluorescein isothiocyanate conjugates for fluorescence purposes; these NPs were used successfully in vitro. However, fluorescein has a strong emission between 500-600 nm, the same wavelength range in which considerable tissue autofluorescence was detected in separate in vivo experiments. This hindered the ability to effectively detect myeloid cells with the NPs in vivo. To remedy this, indocyanine green (ICG), a tricarbocyanine dye that is safe for intravenous administration and approved by the FDA for near-infrared (NIR) optical imaging in humans, was used. ICG has an emission maximum at ˜800 nm, which allows clearer bio-imaging with minimal autofluorescence interference. ICG exhibits negligible fluorescence in aqueous solution and enhanced fluorescence upon interaction with larger molecules such as proteins, making it an ideal candidate for incorporation into albumin NPs. Furthermore, because of its amphiphilic character, ICG loaded in the NPs can effectively simulate the molecular loading of a variety of myeloid-cell-modulating pharmacological small molecules, such as celecoxib or sildenafil, many of which present a combination of hydrophilic and lipophilic properties.

As above, ICG was introduced into the aqueous solution of albumin prior to desolvation, and the process resulted in successful incorporation of ICG into the NPs. After thermal denaturation of NPs and displacement of the medium by PBS, there was no observable leakage of ICG and centrifugal filtration resulted in a completely optically clear supernatant, while the NP-containing pellet had a deep green color (FIG. 1, panel B). The resultant particles had a mean hydrodynamic diameter of 110±4 nm (by intensity distribution, DLS), polydispersity index of 0.24±0.04, ζ-potential of −15.4±0.3 mV (measured in 0.9% NaCl aqueous solution), and they contained 3.3±0.6 wt % ICG. SEM images indicate that dry particles are between 60 and 90 nm in diameter (FIG. 1, panel C).

### Example 2—Nanoparticle Stability and Fluorophore Release

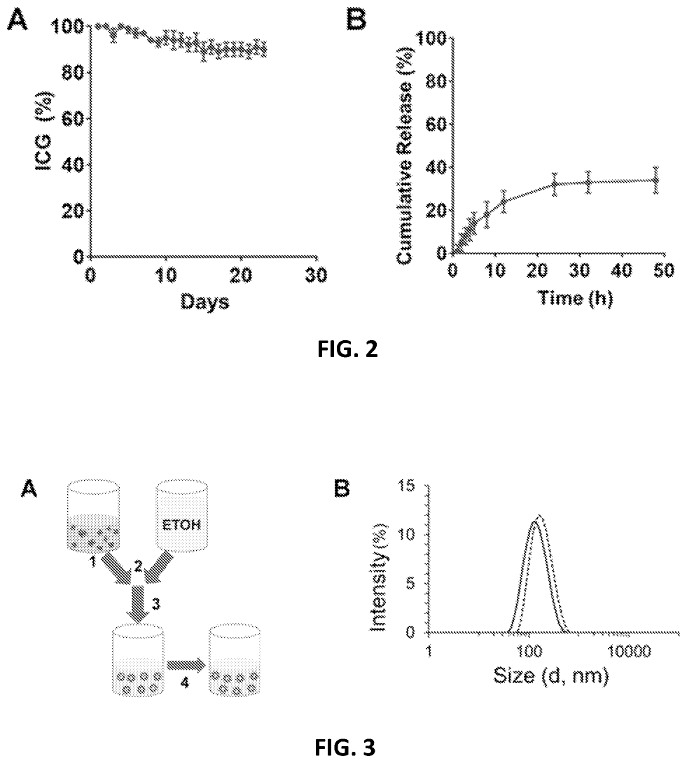

NP stability was evaluated at 4° C. for a month. At this temperature, ICG remained tightly bound to albumin NPs dispersed in PBS (FIG. 2, panel A). The size of these particles was also tracked throughout this period and deviated less than 11% in mean hydrodynamic diameter by intensity as measured by DLS. Strong noncovalent binding stemming from hydrophobic interactions between albumin and ICG likely accounts for this excellent particle stability and negligible fluorophore leakage.

Also measured was the release of ICG at 37° C. in 20% fetal bovine serum (FBS) in PBS solution to mimic physiological conditions. The release of ICG is facilitated by elevated temperature and the presence of proteins from FBS in the release medium (FIG. 2, panel B). Nevertheless, the particles retain more than 80% of their ICG load within 5 hours of exposure to biological medium without the significant initial burst release endemic to many other NP formulations. As shown below, this time frame is sufficient for specific accumulation of the NIR dye in the target cells in vivo for theranostic purposes.

### Example 3—Ligand Conjugation for Targeting Myeloid Cells

Subsequently, G-CSF was covalently linked to the surface of the resultant ICG-loaded NPs using carbodiimide chemistry. G-CSF regulates the activity of myeloid cells and has also been shown to play a crucial role in their generation. Recently, depletion of G-CSF by neutralizing or scavenging immunotherapy was proposed as a possible therapeutic route to decrease the MDSC population. However, the potential side effects of such treatment may include severe neutropenia, vulnerability to infections, and decreased overall anti-cancer immunity. In the present example, alternative strategy was employed. In particular, G-CSF was employed as a ligand to target ICG-loaded NPs to myeloid cells, exploiting the abundance of G-CSF receptors on the myeloid cell surface and the preservation of ligand-receptor specificity upon N-terminal ligation of G-CSF. Such a strategy is compatible with anti-G-CSF treatment or other immunotherapies. The process of G-CSF-conjugated particle formation is schematically illustrated in FIG. 3, panel A. Carbodiimide linkage of G-CSF led to a slight increase (22±3%) in the mean hydrodynamic diameter of the resultant NPs, resulting in 141±4 nm diameter with a polydispersity index of 0.17±0.05 (FIG. 3, panel B). The conjugated NPs exhibited excellent storage stability in PBS at 4° C. for 5 weeks with fluctuations in the mean hydrodynamic diameter of less than 9%. They were used for biological experiments within this period of time.

### Example 4—Myeloid Cell Uptake

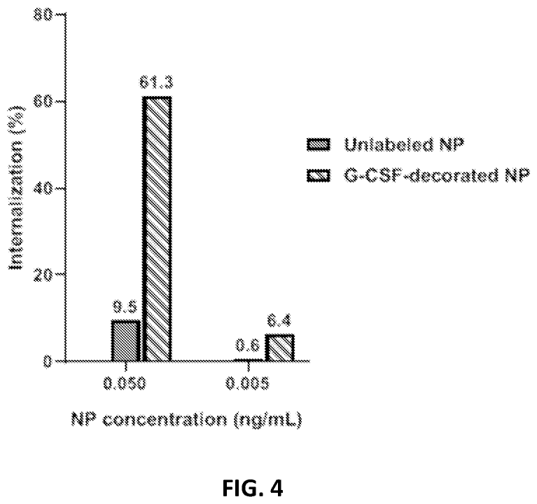

The effect of the G-CSF decoration of NPs was tested in a preliminary experiment conducted in the RAW264.7 cell line, a macrophage cell line derived from multiple myeloma that consistently expresses the G-CSF receptor (CSF3R). Dispersions of 0.05 ng/mL or 0.005 ng/mL decorated or control (undecorated) NPs containing albumin-fluorescein isothiocyanate conjugate were added to the RAW264.7 cells diluted to 500,000 cells per well. Flow cytometry characterization of cells revealed that G-CSF decoration of the NPs greatly enhanced their uptake into RAW264.7 cells at each dose tested (FIG. 4).

### Example 5—Uptake in Mixed Primary Murine Splenocytes

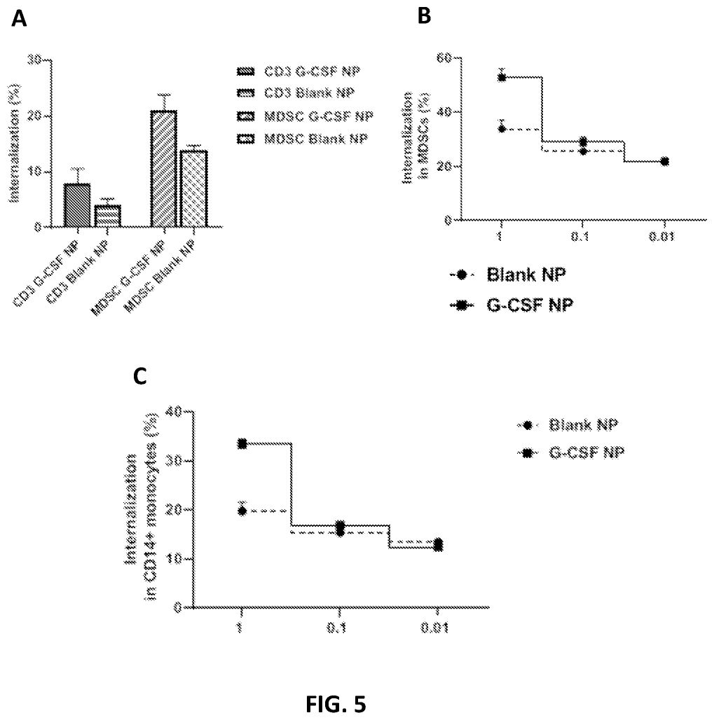

NP uptake specificity was assessed in vitro in mixed primary murine splenocytes containing T-cells, B-cells, NK cells, macrophages, neutrophils, dendritic cells, and MDSCs isolated from both tumor-free and 4T1 triple negative breast cancer-bearing mice. MDSCs are normally a rare cell population at <5% of circulating peripheral blood mononuclear cells (PBMCs). The 4T1 murine breast cancer model reliably increases MDSC populations in the blood, spleen, and tumor microenvironment within 10 days of implantation. Thus, 4T1 tumor-bearing mouse splenocytes contain many more MDSCs than tumor-free mice. Mixed splenocytes were exposed for 30 minutes to various concentrations of NPs in media. Across all experiments and conditions, G-CSF decoration of the NPs greatly increased myeloid cell uptake, including uptake into MDSCs (FIG. 5). Limited nonspecific uptake into less than 10% of total CD3+ T-cells was also observed, although G-CSF decoration did not significantly increase NP uptake into CD3+ cells, suggesting that nonspecific uptake of albumin NPs occurs in a limited proportion of cells. Uptake of these NPs into MDSCs was significantly higher (P=0.0044), and G-CSF decoration increased the uptake (FIG. 5, panel A). It can be seen in FIG. 5 (panel B) that decorated NPs achieved above 50% accumulation in MDSCs and above 30% accumulation in CD14+ non-MDSC monocytes (FIG. 5, panel C), showing differential accumulation and the potential to preferentially label or alter myeloid cells. NP uptake was defined as cells emitting fluorescence at >800 nm as a result of ICG internalization.

### Example 6—Nanoparticle Uptake In Vivo

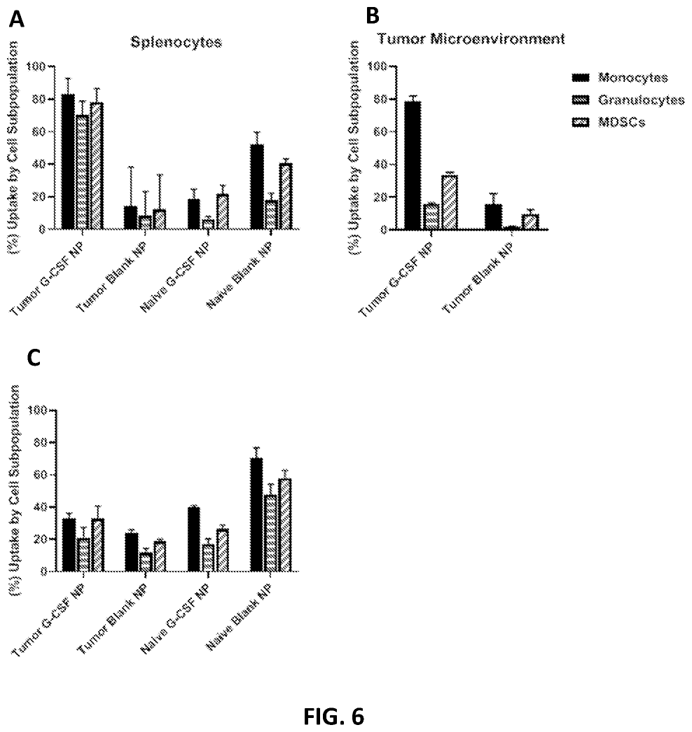

The effect of G-CSF decoration of NPs on cell specificity in vivo was tested in 4T1 tumor-bearing mice. A 10 μg bolus dose of ICG-loaded NPs suspended in 100 μL sterile PBS was injected via tail vein in anesthetized mice and the NP distribution was then imaged using the Lago X (Spectral Instruments Imaging) in vivo imaging system. Images were taken at 0, 30, and 180 minutes after NP administration. At 180 minutes, mice were sacrificed to harvest spleen, liver, and tumor. Harvested tissues were processed into single-cell suspensions, which were then fixed and prepared for flow cytometry. Results showed that G-CSF-decorated NPs accumulated in MDSCs at significantly higher rates compared to undecorated (blank) NPs in the spleen and tumor microenvironment (P=0.0076 and P=0.0002 respectively, FIG. 6, panels A and B). On the other hand, 4T1 tumor-bearing mouse livers took up all NPs at significantly lower rates than did tumor-free mouse livers, which displayed increased uptake of nanoparticles regardless of surface decoration; this finding is likely indicative of homeostatic reticuloendothelial system activity that is altered in tumor-bearing hosts and potentially is caused by lower amounts of NP available for uptake due to MDSC uptake elsewhere (FIG. 6, panel C). These data demonstrate that G-CSF decoration of albumin NPs is an effective strategy to achieve preferential myeloid and MDSC accumulation in vivo where specificity effects are enhanced in the tumor-bearing mice vs. tumor-free mice. Furthermore, these data also indicate that albumin NPs can effectively circulate through the bloodstream into myeloid cells within the tumor microenvironment, as shown by nanoparticle accumulation in ˜70% of monocytes and ˜35% of MDSCs isolated from the 4T1 tumor microenvironment.

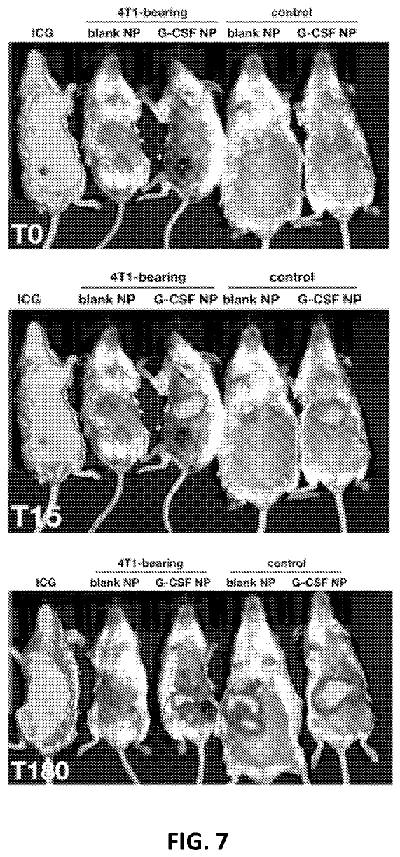

Improving the selectivity of myeloid tracking and reprogramming is a key to effective treatment of immunologically active cancers and other conditions. Shown in the present examples is that decoration of small molecule-bearing albumin NPs with recombinant G-CSF is not only reproducibly formulable and stable in a variety of conditions, but also increases preferential uptake into myeloid cells both in vitro in a cell line and in mixed immune cells from both tumor-naïve and 4T1 metastatic triple-negative breast cancer-bearing mice (in which MDSCs and other immunosuppressive myeloid cells play a deleterious role by promoting disease progression and treatment resistance). This trend was also observed in vivo, where G-CSF-decorated NPs effectively accumulated in myeloid cells in the liver, spleen, and tumor microenvironment within just 3 hours at much greater rates than control undecorated NPs, which displayed nonspecific uptake regardless of tumor condition. Surprisingly, G-CSF decoration significantly decreased NP uptake in the liver and spleen of nontumor-bearing animals, suggesting that G-CSF decoration could drive decreased nonspecific nanoparticle uptake by the Kupffer cells and splenocytes of the reticuloendothelial system in a tumor-free model and is an effective strategy to ensure concentration of NPs within the tumor microenvironment (particularly with repeated dosing schedules). By achieving accumulation in more than 50% of all monocytes and more than 20% of MDSCs in the 4T1 tumor microenvironment, G-CSF-decorated NPs provide substantial insight into the levels of local myeloid cell presence via accumulation in a significant proportion thereof in a single dose. All NPs tested in vivo were labeled with ICG, providing an optimized strategy to assess cell subpopulation uptake and overall distribution throughout the body via the Lago X imaging system (FIG. 7). The stimulated cells were treated with 0.01 mg Celecoxib-loaded ICG albumin NPs.

### Example 7—Celecoxib-NPs Reduce Reactive Oxygen Species (ROS) In Vitro

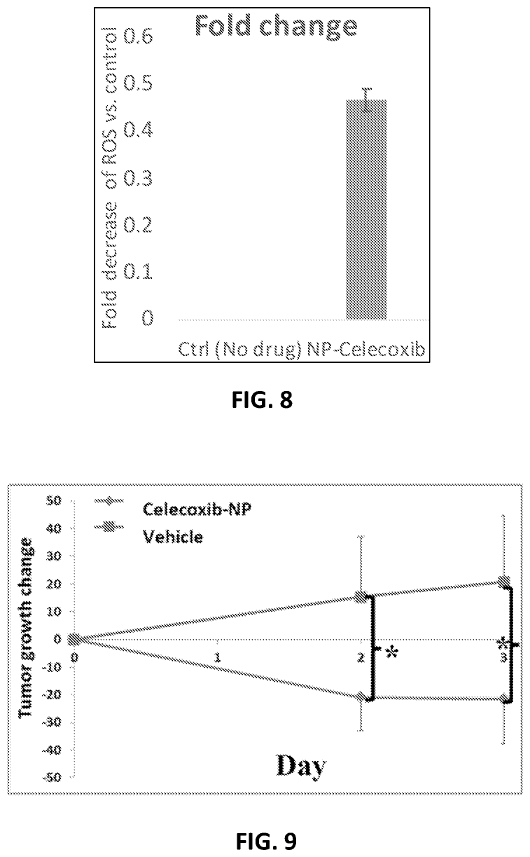

In this example, RAW 264.7 macrophages were LPS-stimulated to induce an immunosuppressive phenotype. The LPS-stimulated RAW cells were treated with 0.01 mg Celecoxib-loaded ICG albumin NPs. The Celecoxib-NPs significantly reduced (by 47%) ROS produced by immunosuppressive RAW cells based on flow cytometry (fluorescent ROS assay), suggesting decreased immuno-suppressive activity (FIG. 8). These data show that Celecoxib-NPs improve inhibition of immunosuppression in vitro, which likely supports the observed significant in vivo tumor shrinkage (see FIG. 9).

### Example 8—Celecoxib-Loaded MTN Reduce Tumor Burden

In this example, Celecoxib-loaded albumin NPs were tested for drug retention. The NPs exhibited drug retention in 20% serum at 37° C. for extended periods of time (FIG. 3). The results verify that Celecoxib-albumin NPs will not rapidly break apart and fail to deliver payload when exposed to the in vivo environment. A single intravenous injection of Celecoxib-loaded albumin NPs significantly reduced 4T1 tumor size in N=6 tumors/group (FIG. 9), yielding robust evidence of the efficacy of the albumin NP platform.

The data collectively suggest G-CSF-NPs target myeloid cells (particularly MDSCs) and reduce immunosuppression, that G-CSF-NPs target tumors, and that treatment with G-CSF-NPs reduce tumor size.

### Materials and Methods

Materials

BSA (average mw 66.0 kDa, ≥96%) was purchased from Sigma-Aldrich, BSA-fluorescein isothiocyanate (FITC) conjugate with molar ratio of FITC/BSA≥7 and BSA average mw 66.0 kDa was obtained from Sigma-Aldrich, ICG was from Sigma, recombinant murine G-CSF (19.0 kDa) was obtained from PeproTech, PBS (1×, pH 7.4), sodium hydroxide 1N, ethanol absolute, and biological grade water were purchased from Fisher Scientific.

BSA Nanoparticle Formation and ICG Loading

NPs were formed by dissolving BSA 5 wt % in water adjusted to pH=8.5 by NaOH, with subsequent desolvation of albumin by gradual addition of ethanol 1:4 v/v. To stabilize the NPs and prevent albumin dissociation, the resultant mixture was subjected to 3 rounds of mild heating (70° C. for 10 minutes) to denature free albumin chains within the NPs. To load the NPs with ICG, an excess of ICG (2 wt %) was dissolved in water at pH=8.5 with BSA as above. The desolvation and particle denaturation steps above were unchanged. The NPs with incorporated ICG were then washed with an excess of PBS to remove unincorporated dye and to displace the medium with a biologically compatible one. Washing was performed by centrifugal filtration at 10,000 rpm for 5 min in an Eppendorf 5415 C Centrifuge using a 100 kDa MWCO EMD Millipore Amicon™ Ultra-0.5 centrifugal filter units. Washing was repeated thrice, resulting in a completely clear filtrate, and the nanoparticle-containing pellet was deep green. NPs were stored at 4° C., protected from light.

For the formation of BSA-fluorescein isothiocyanate (FITC) NPs in the preliminary experiments (FIG. 4), BSA-FITC conjugate was dissolved at 5 wt % in PBS. This was followed by a gradual desolvation/heat denaturation process when overall 170 wt % of anhydrous ethanol was added to the solution followed by several short (overall 15 min) NP denaturation cycles at 70° C. NPs were then washed with excess PBS by centrifugal filtration as described above, stored at 4° C., and protected from light.

Nanoparticle Characterization

NP size was assessed in PBS dispersion by dynamic light scattering (DLS). The size distribution was measured at room temperature using a Nano-ZS90 Zetasizer (Malvern, U.K.) equipped with a 632.8 nm laser source. The same instrument was used to evaluate NP ζ-potential, however for these measurements the media was replaced with 0.9% water solution of NaCl to avoid affecting electrophoretic mobility by the high concentration of electrolytes in the media. Each measurement was performed in triplicate in three independent samples.

The size and morphology of the resultant dry NPs was corroborated by high-resolution SEM. For these measurements, the PBS in the medium was replaced with deionized water by three washes using centrifugal filtration as above, and the resultant dispersion was diluted 1:50 with deionized water. It was then deposited onto 15 mm aluminum SEM stubs and allowed to air-dry overnight. All samples were gold-palladium sputter-coated with a Denton Desk II sputter-coater (Denton Vacuum, Moorestown, N.J.). SEM imaging was carried out by a high-resolution Zeiss Sigma field emission scanning electron microscope (FESEM) (Zeiss Microscopy, Thornwood, N.Y.) operated at an accelerating voltage of 2-3 kV using InLens Secondary Electron (SE) detection, and 5-7 kV using Backscattered Electron Detection (BSD). Images were captured in TIFF using storage resolution 2048×1536 pixels and a line averaging noise reduction algorithm.

ICG concentration was determined by UV-vis spectroscopy using an absorbance microplate reader (Azure Biosystems, Dublin, USA) with a 750 nm filter. Specifically, the ICG-loaded NP dispersion after washing, or the filtrate collected from centrifugal filtration, was diluted with known excess volumes of 50% v/v aqueous DMSO solution and sonicated for 15 min to extract the ICG from denatured albumin. Absorbance was then measured and the concentration was calculated using a standard calibration curve for ICG. To assess particle stability, the concentration of ICG was monitored by aliquoting the dispersion, performing centrifugal filtration as above, and measuring the ICG concentration in the pellet. Similarly, for release experiments, the ICG content inside the dialysis tubes was determined from independent samples exposed to release for different time intervals. This minimized the potential error of free ICG degradation in aqueous solution over time. Nanoparticle fluorescence was monitored using a TECAN infinite M1000 plate reader (Männedorf, Switzerland) and fluorescence microscope (DMRXE, Leica, Germany).

G-CSF Conjugation

The NPs were covalently linked to G-CSF using carbodiimide chemistry based on amide bond formation between BSA chains and G-CSF protein under mild aqueous conditions with a zero-length crosslinker that leaves no residues in the resultant protein. Briefly, a 20 μg/100 μL solution of G-CSF was activated with 5 mM of EDC (1-ethyl-3-(3-dimethylaminopropyl) carbodiimide hydrochloride)/Sulfo-NHS (sulfo-N-hydroxysuccinimide) at pH=5.5 and the NP dispersion medium was adjusted to pH 5.5 by NaOH addition. Both solutions were then united and stirred at 4° C. for 4 hours protected from light. The approximate molar mixed ratio between G-CSF and NPs was 10:1. NPs were then dialyzed against PBS for 18 h at 4° C. using Pur-A-Lyzer™ dialysis tubes (MWCO 6-8 kDa, Sigma-Aldrich) protected from light.

RAW264.7 Internalization

The RAW264.7 myeloid cell line was cultured in standard Dulbecco's Modified Eagle Medium (DMEM) with 10% Fetal Bovine Serum. RAW264.7 cells at 70% confluency were spun down, washed in PBS, diluted to 500,000 cells per well in a round-bottom 96-well plate, and then were added to standard DMEM containing 0.05 ng/mL or 0.005 ng/mL decorated or control (undecorated) NPs containing albumin-fluorescein isothiocyanate conjugate. Cells with NPs were incubated at 37° C. for 90 minutes then spun down at 1100 rpm for 3 minutes to aspirate supernatant containing noninternalized NPs, washed once in PBS to remove remaining noninternalized NPs, and then fixed in 5% formalin, and stained with propidium iodide. NP uptake was then quantified via flow cytometry as described below.

Bulk Splenocyte Uptake

Bulk splenocytes containing mixed T-cells, B-cells, and myeloid cells were isolated from wild-type Balb/c mice via surgical harvest of the spleen in both 4T1 triple negative breast cancer-bearing mice—which is known to induce more MDSCs—and tumor-free mice. Splenocytes were extracted via maceration of the spleen in PBS followed by filtration through a 70 μm nylon cell filter, washing the well in which the spleen was macerated with PBS through the cell filter up to 20 mL to increase yield. Cells were then spun down at 1100 rpm for 3 minutes, followed by removal of the supernatant and incubation in 5 mL of ACK Lysis Buffer at room temperature for 5 minutes. Subsequently, the cells were spun down again and resuspended in PBS for quantification of cell yield via hemacytometer. Bulk splenocytes were then plated into 96-well round-bottom plates in standard Roswell Park Memorial Institute (RMPI) culture medium at 250,000-500,000 cells per well. Aliquots containing ICG-labeled NPs were prepared at 2× their final concentration (1 μg, 100 ng, 10 ng) in RPMI and then diluted 1:2 in each well of a 96 well plate. NPs tested included non-G-CSF-decorated NPs as well as G-CSF-decorated NPs. Splenocytes were incubated at 37° C. in RPMI containing NPs for 30 minutes, after which they were spun down, washed once in PBS, and resuspended in 10% formalin for fixation and flow cytometric characterization.

Tumor Microenvironment Study

4T1 tumor-bearing mice were prepared via subcutaneous implantation of 10⁵ 4T1 cells in the right mammary flank of female Balb/c mice and allowed to grow for 10 days, by which point tumors were palpable and ˜1 cm in diameter. Mice were then anesthetized via isofluorane inhalation and placed in the imaging chamber of a Lago X, Spectral Instruments Imaging) and administered 10 μg of ICG-labeled NPs suspended in 100 μL of sterile PBS via tail vein. Both G-CSF-decorated NPs and undecorated NPs were tested. Lago X images were taken at the time of administration and again at 3.5 hours post-injection, at which point mice were sacrificed. Then tumor, liver, and spleen were harvested. Each tissue was macerated, with tumor enzymatically digested in collagenase IV solution, to prepare single-cell suspensions that were then fixed in 10% formalin prior to flow cytometric analysis.

Flow Cytometry

Fixed cells suspended in PBS were spun down at 1100 rpm for 3 minutes and resuspended in blocking buffer (3% bovine serum albumin, 10% fetal bovine serum in PBS) and incubated on ice for 30 minutes. Dilutions of fluorophore-conjugated commercially-available antibodies (Bio-Rad, Lonza, Thermo-Fisher) were prepared in the same buffer. Fixed cells were spun down, supernatant removed, and antibodies were then incubated on ice for another 30 minutes. Cells were then spun down and washed in PBS three times prior to flow cytometric analysis on a Luminex Guava easyCyte cytometer. Flow cytometry was performed within 4 days of fixing cells. All analysis was done with FloJo, where NP-positive cells were counted as those emitting NIR fluorescence from ICG-NPs as a percentage of all counted cells, not as an absolute quantification of NPs per cell.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A targeted nanoparticle comprising:
a nanoparticle; and
a myeloid cell (MC) targeting moiety stably associated with the outer surface of the nanoparticle.

2. The targeted nanoparticle of claim 1, wherein the greatest dimension of the nanoparticle is from 10 to 200 nm.

3. The targeted nanoparticle of claim 1, wherein the greatest dimension of the nanoparticle is from 30 to 100 nm.

4. The targeted nanoparticle of claim 1, wherein the nanoparticle is spherical or spheroid.

5. The targeted nanoparticle of claim 1, wherein the nanoparticle is a protein nanoparticle.

6. The targeted nanoparticle of claim 5, wherein the nanoparticle is a serum protein nanoparticle.

7. The targeted nanoparticle of claim 6, wherein the nanoparticle is an albumin protein nanoparticle.

8. The targeted nanoparticle of claim 7, wherein the albumin protein nanoparticle comprises an albumin protein selected from the group consisting of: bovine serum albumin (BSA), human serum albumin (HSA), polymerized bovine serum albumin (pBSA), polymerized human serum albumin (pHSA), recombinant albumin, Albumin-DX LR, and any combination thereof.

9. The targeted nanoparticle of any one of claim 1, wherein the MC targeting moiety is selected from the group consisting of: a polypeptide, an antibody, a ligand, an aptamer, a nanoparticle, and a small molecule.

10. The targeted nanoparticle of any one of claim 1, wherein the MC targeting moiety binds to a molecule on the surface of MCs.

11. The targeted nanoparticle of claim 10, wherein the MC targeting moiety binds to a receptor on the surface of MCs.

12. The targeted nanoparticle of any one of claim 1, wherein the MC targeting moiety is an immunosuppressive myeloid cell (isMC) targeting moiety.

13. The targeted nanoparticle of claim 12, wherein the isMC targeting moiety is a ligand.

14. The targeted nanoparticle of claim 13, wherein the isMC targeting moiety is granulocyte-colony stimulating factor (G-CSF).

15. The targeted nanoparticle of any one of claim 1, wherein the MC targeting moiety is stably associated with the outer surface of the nanoparticle via an amide bond.

16. The targeted nanoparticle of any one of claim 1, further comprising a detectable label.

17. The targeted nanoparticle of claim 16, wherein the detectable label is an in vivo imaging agent.

18. The targeted nanoparticle of claim 17, wherein the in vivo imaging agent is a near-infrared (NIR) imaging agent.

19. The targeted nanoparticle of claim 17, wherein the in vivo imaging agent is a photoacoustic imaging agent.

20. The targeted nanoparticle of any one of claim 17, wherein the in vivo imaging agent is indocyanine green (ICG).

21. The targeted nanoparticle of any one of claim 16, wherein the detectable label is incorporated into the nanoparticle.

22. The targeted nanoparticle of any one of claim 16, wherein the detectable label is stably associated with the outer surface of the nanoparticle.

23. The targeted nanoparticle of any one of claim 1, further comprising a drug.

24. The targeted nanoparticle of claim 23, wherein the drug is a small molecule drug.

25. The targeted nanoparticle of claim 23 or claim 24, wherein the drug is an isMC-modulating drug.

26. The targeted nanoparticle of claim 25, wherein the drug is an isMC-disrupting drug.

27. The targeted nanoparticle of claim 26, wherein the drug is an isMC metabolism-disrupting drug.

28. The targeted nanoparticle of claim 27, wherein the drug is a nonsteroidal anti-inflammatory drug (NSAID).

29. The targeted nanoparticle of claim 28, wherein the NSAID is a cyclooxygenase-2 (COX-2) inhibitor.

30. The targeted nanoparticle of claim 29, wherein the COX-2 inhibitor is celecoxib.

31. The targeted nanoparticle of claim 25, wherein the drug is sildenafil.

32. The targeted nanoparticle of any one of claim 23, wherein the drug is releasably incorporated into the nanoparticle.

33. The targeted nanoparticle of any one of claim 23, wherein the drug is releasably associated with the outer surface of the nanoparticle.

34. A composition comprising targeted nanoparticles of any one of claims 1 to 33.

35. The composition of claim 34, wherein the composition is a pharmaceutical composition comprising the targeted nanoparticles and a pharmaceutically acceptable carrier.

36. The composition of claim 35, wherein the pharmaceutical composition comprises targeted nanoparticles of any one of claims 17 to 22.

37. A method of imaging myeloid cells (MCs) in a subject, comprising:
administering to the subject the pharmaceutical composition of claim 36 in an amount effective to image MCs in the subject; and
imaging MCs in the subject by in vivo imaging.

38. The method according to claim 37, wherein the targeted nanoparticles comprise an isMC targeting moiety.

39. The method according to claim 38, wherein the isMC targeting moiety is a ligand.

40. The method according to claim 39, wherein the isMC targeting moiety is G-CSF.

41. The method according to any one of claim 38, wherein the subject comprises a tumor, and wherein the method comprises assessing infiltration of the isMCs in the microenvironment of the tumor based on the imaging of the isMCs.

42. The method according to claim 41, further comprising providing a diagnosis, prognosis, or both, of the subject based on the imaging of the isMCs.

43. The method according to any one of claim 37, wherein the pharmaceutical composition comprises the targeted nanoparticles of any one of claims 19 to 22, and wherein the in vivo imaging comprises photoacoustic imaging.

44. The method according to any one of claim 37, wherein the targeted nanoparticles further comprise a drug as defined in any one of claims 23 to 33.

45. A method of modulating MCs in a subject, comprising administering to the subject a pharmaceutical composition comprising:
targeted nanoparticles of claim 25; and
a pharmaceutically acceptable carrier,
in an amount effective to modulate MCs in the subject.

46. A method of enhancing an anti-tumor immune response in a subject having a tumor, comprising administering to the subject a pharmaceutical composition comprising:
targeted nanoparticles of claim 25; and
a pharmaceutically acceptable carrier,
in an amount effective to disrupt MCs in the subject.

47. The method according to claim 45 or claim 46, wherein the MCs are isMCs and the targeted nanoparticles comprise an isMC targeting moiety.

48. The method according to claim 47, wherein the isMC targeting moiety is a ligand.

49. The method according to claim 48, wherein the isMC targeting moiety is G-CSF.

50. The method according to claim 45 or claim 46, wherein the targeted nanoparticles comprise a detectable label as defined in claim 17.

51. The method according to claim 50, further comprising imaging the MCs in the subject.

52. A kit, comprising:
the composition of claim 34; and
instructions for targeting the targeted nanoparticles to MCs.

53. A kit, comprising:
the pharmaceutical composition of claim 35; and
instructions for administering the pharmaceutical composition to a subject to target the targeted nanoparticles to MCs in the subject.
